(19) United States
(12) Patent Application Publication
(10) Pub. No.: US 2023/0363707 A1
(43) Pub. Date: Nov. 16, 2023

(54) DEVICES AND RELATED METHODS FOR PHYSIOLOGICAL EXAMINATION

(71) Applicant: THE REGENTS OF THE UNIVERSITY OF CALIFORNIA, Oakland, CA (US)

(72) Inventors: Cody Dunn, Irvine, CA (US); Bernard Choi, Irvine, CA (US); Christian Crouzet, Irvine, CA (US)

(21) Appl. No.: 18/246,636

(22) PCT Filed: Sep. 27, 2021
(86) PCT No.: PCT/US21/52161

§ 371 Date: Mar. 24, 2023

Related U.S. Application Data
(60) Provisional application No. 63/083,361, filed on Sep. 25, 2020.

(51) Int. Cl. A61B 5/00

(52) U.S. Cl. CPC A61B 5/4866; A61B 5/6801; A61B 5/0059

(57) ABSTRACT

Provided herein are devices and related methods that may be used for a physiological examination. In one embodiment, a device that may be used for physiological examination comprising a detector with a slit aperture and a light source, so that the detector comprises a lens that enables a wider field of view but does not require an image to be in focus, in one embodiment, a method of diagnosing a disease in a subject through physiological examination using a device comprising a detector with a slit aperture and a plurality of light sources, including different wavelength LEDs and/or vertical-cavity surface-emitting lasers (VCSELs).

### CROSS-REFERENCES TO RELATED APPLICATIONS

This application claims benefit of U.S. Provisional Application No. 63/083,361 filed Sep. 25, 2020, the specification of which is incorporated herein in its entirety by reference.

### FIELD OF THE INVENTION

The present disclosure relates to the field of health care and specifically physiological examination.

### BACKGROUND OF THE INVENTION

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Tissue metabolic rate of oxygen consumption (tMRO2) is a clinical marker for many pathologies such as peripheral arterial disease, cancer, and overall cardiovascular health. However, the development of a sensor that provides the appropriate metrics for continuous monitoring of tMRO2 has been challenging. Some limitations are that it has proved difficult to develop such a sensor that is inexpensive, noninvasive, and/or wearable. For example, an optical technique for estimating tMRO2 using a combination of diffuse correlation spectroscopy (DCS) and diffuse optical spectroscopic imaging (DOSI) has been prohibitively expensive and bulky. Or, for example, a wide field optical technique using coherent spatial frequency domain imaging (cSFDI) for noncontact monitoring of tMRO2 is limited in its effectiveness as it requires physiological assumptions such as arterial oxygen saturation of 100% and is highly susceptible to motion artifact.

Thus, there is a need in the art for novel and effective devices and related methods for physiological examination, including the examination of clinical markers that may include, but is in no way limited to, tMRO2.

### BRIEF SUMMARY OF THE INVENTION

It is an objective of the present invention to provide devices and methods that allow for the examination of clinical markers that may include, but is in no way limited to, tMRO2, as specified in the independent claims. Embodiments of the invention are given in the dependent claims. Embodiments of the present invention can be freely combined with each other if they are not mutually exclusive.

The present invention features a device used for physiological examination of a subject, comprising a detector and one or more light sources. The detector may comprise a camera with a lens and the light sources may comprise one or more coherent light sources or a combination of multiple light sources (including, but not limited to LEDs and VCSELs). The detector may include an aperture for point estimation of optical properties for the device in the front of the detector. The device may further comprise circuitry to allow rapid switching between wavelengths of the plurality of light sources. The device may be configured to be used as a sensor that is inexpensive to manufacture, noninvasive, and/or wearable for a user. In another embodiment, the device may provide measurements of blood flow, oxygenation, and/or metabolism. In another embodiment, the device further comprises a program for real-time processing of collected images. In one embodiment, a device for physiological examination includes examination of one or more clinical markers may provide measurements of blood flow, oxygenation, and/or metabolism. In some embodiments, the one or more clinical markers comprise tissue metabolic rate of oxygen consumption (tMRO2), tMRO2 is a clinical marker for many pathologies such as peripheral arterial disease, cancer, and overall cardiovascular health and thus, the various embodiments of the device and related methods may also be used for the monitoring, detection, diagnosing, prognosing, and treatment of various pathologies including peripheral arterial disease, cancer, and overall cardiovascular health.

One of the unique and inventive technical features of the present invention is the implementation of a detector comprising an aperture in a device for detecting clinical markers. Without wishing to limit the invention to any theory or mechanism, it is believed that the technical feature of the present invention advantageously provides for compact and time-efficient methods of diagnosing a plurality of physiological states including peripheral arterial disease, cancer, and overall cardiovascular health. None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows, in accordance with an embodiment herein, a device comprising a detector, a light source, and a circuitry for detector control. The device is depicted as placed on a silicone phantom for measurement.

FIG. 2 shows, in accordance with an embodiment herein, a sensor with a source and slit. In accordance with various embodiments herein, the bottom of the sensor is depicted which should be placed in contact with a subject for measurements.

FIG. 3 shows, in accordance with an embodiment herein, a circuitry for use in conjunction with a device comprising a detector and a light source. In one embodiment, the circuitry is used for powering the source. In another embodiment, modifications will enable rapid switching between wavelengths.

FIG. 4 shows, in accordance with an embodiment herein, an example of a holder that was produced through 3D printing methods, that may be used for holding a device comprising a detector and light source. In one embodiment, additional light sources (including, but not limited to LEDs and VCSELs) placed in the holes may improve the stability and accuracy of the sensor by increasing the number of source-detector separations. In another embodiment, the removal of these holes may allow for smaller sensors.

FIG. 5 shows, in accordance with an embodiment herein, an unprocessed camera image taken from a phantom at a single wavelength.

FIG. 6 shows, in accordance with an embodiment herein, a flowchart of an example of a potential processing scheme to obtain physiological parameters.

FIGS. 7A-7I show, in accordance with an embodiment herein, a sequence of plots representing in vitro (silicone phantom) and in vivo (human finger) preliminary data taken at three different wavelengths (680 nm, 775 nm, 850 nm) using a device comprising a detector and light source. The plots from the in vitro tests demonstrate the ability to differentiate between phantoms of different optical properties when compared to Virtual Photonics simulations. The plots from the in vivo tests demonstrate the ability to detect the SPG and PPG and waveforms. Further, in one embodiment, reduction of motion artifact and increased filtering could be used to improve signal quality. In another embodiment, additional holes can be placed to assist with the identification of which light source is on at any time point during data collection. Specifically, FIG. 7A shows a plot of R(p) [mm-2] versus p [mm] in a plurality of different models at 680 nm. FIG. 7B shows a graph of various device measurements corresponding to colors in simulations at 680 nm. FIG. 7C shows a graph of SPG and PPG measurements over time at 680 nm. FIG. 7D shows a plot of R(p) [mm-2] versus p [mm] in a plurality of different models at 775 nm. FIG. 7E shows a graph of various device measurements corresponding to colors in simulations at 775 nm. FIG. 7F shows a graph of SPG and PPG measurements over time at 775 nm. FIG. 7G shows a plot of R(p) [mm-2] versus p [mm] in a plurality of different models at 850 nm. FIG. 7H shows a graph of various device measurements corresponding to colors in simulations at 850 nm. FIG. 7I shows a graph of SPG and PPG measurements overtime at 850 nm.

FIGS. 8A-8F shows, in accordance with an embodiment herein, a sequence of plots representing in vivo (human finger) preliminary data taken with a device comprising a detector and light source with updated circuitry that switches between three different wavelengths (680 nm, 775 nm, 850 nm). As depicted, the camera acquires images at approximately 135 frames per second. Thus, each wavelength was acquired at approximately 45 frames per second. The plots from the in vivo tests demonstrate the ability to detect changes in blood flow, deoxyhemoglobin, and oxyhemoglobin. Specifically, FIG. 8A shows a photograph of an embodiment of the present invention. FIG. 8B shows a schematic of an embodiment of the present invention. FIG. 8C shows a graph of unfiltered blood flow trends during an occlusion match. FIG. 8D shows a graph of zoomed-in blood flow to see an SPG signal. FIG. 8E shows a first plot of measured oxyhemoglobin levels. FIG. 8F shows a second plot of measured oxyhemoglobin levels.

FIG. 9A shows a photograph and 3D model of the CSI sensor probe of the present invention. FIG. 9B shows a block diagram of the timing scheme used to control the plurality of light sources (VCSEL and LEDs). FIG. 9C shows representative measurements of the VCSEL (850 nm) and LEDs.

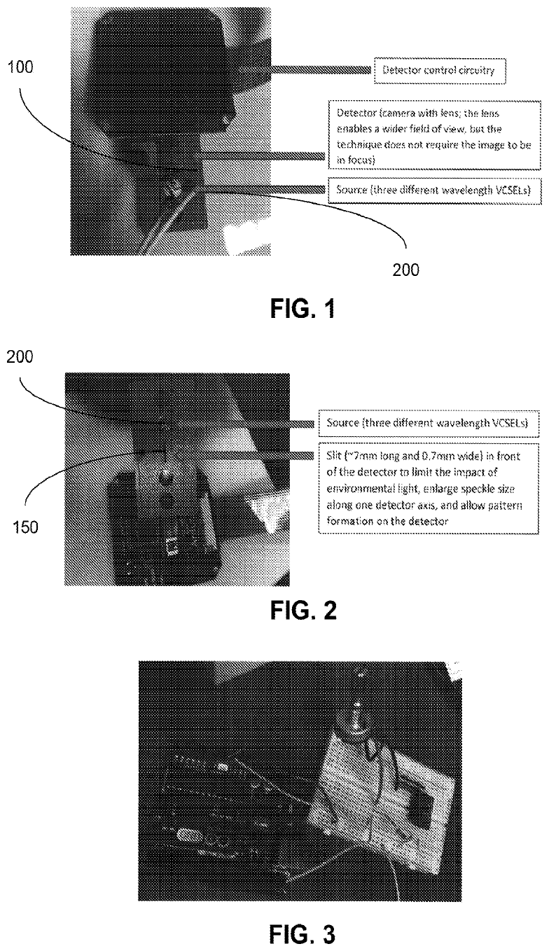

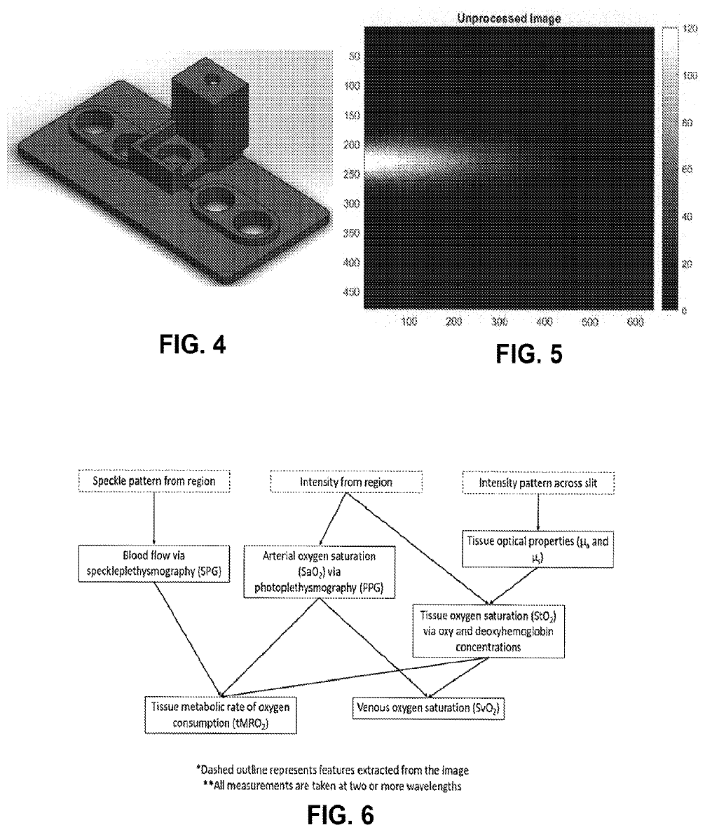

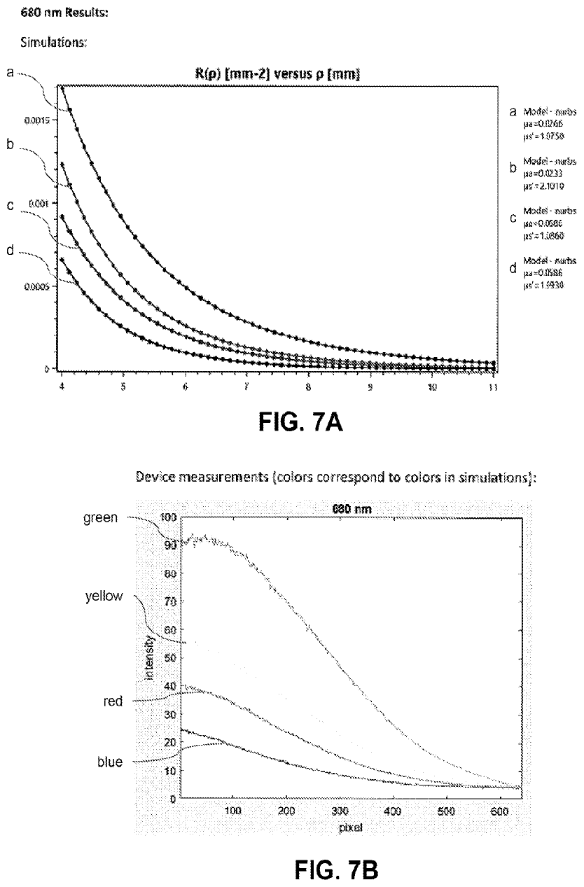

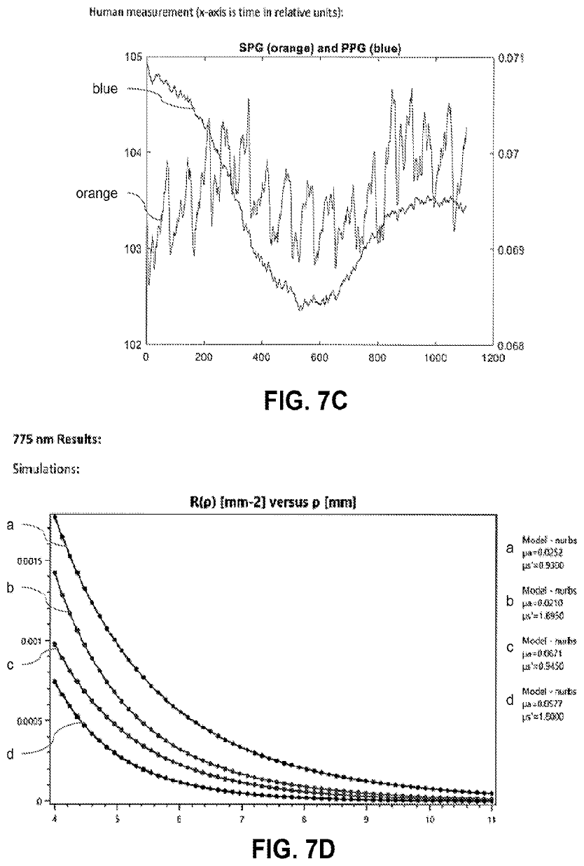

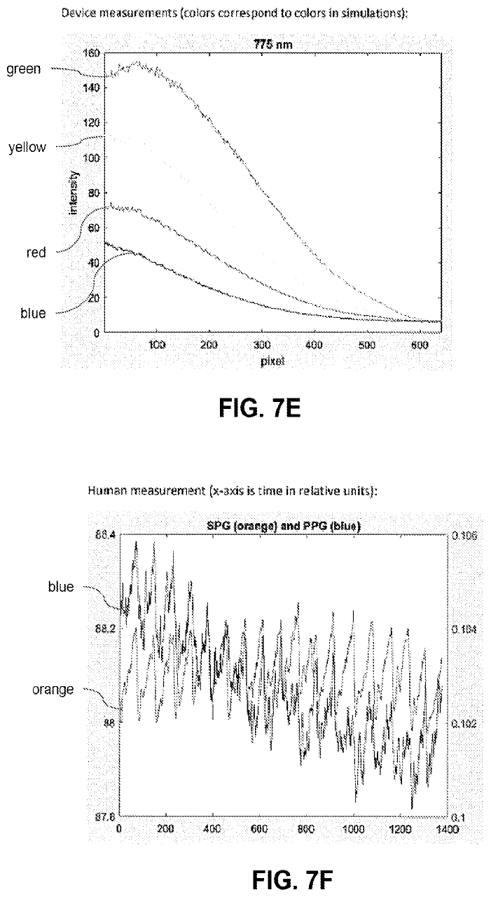

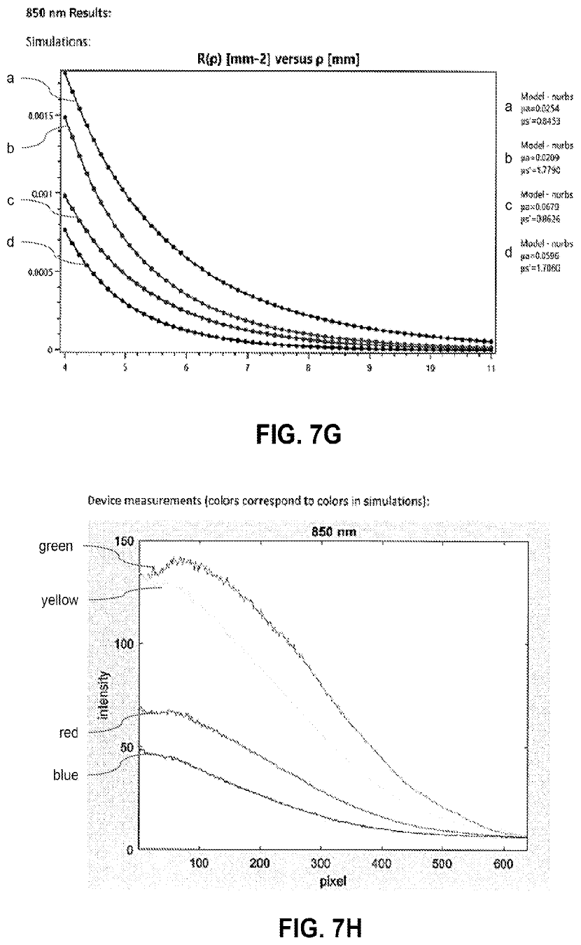

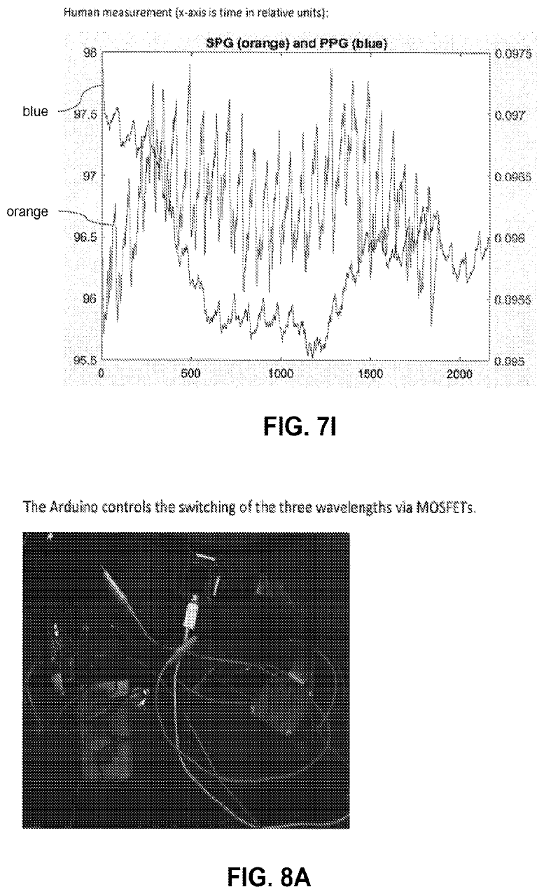

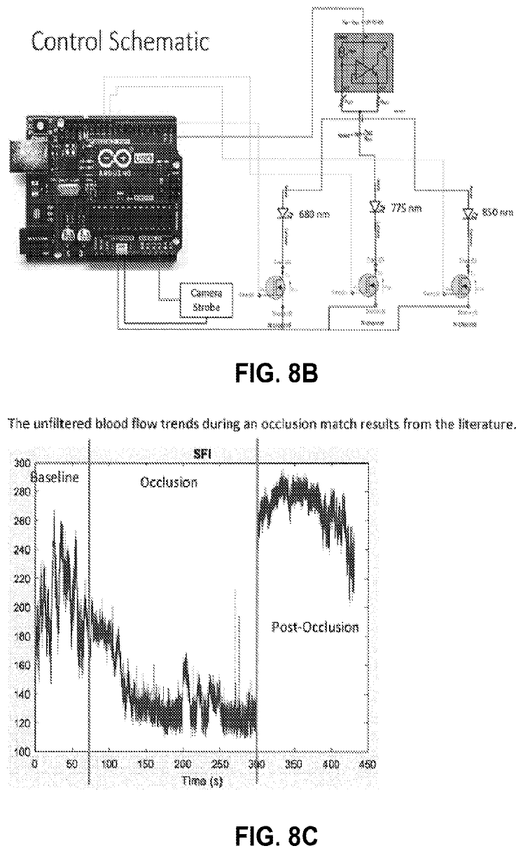

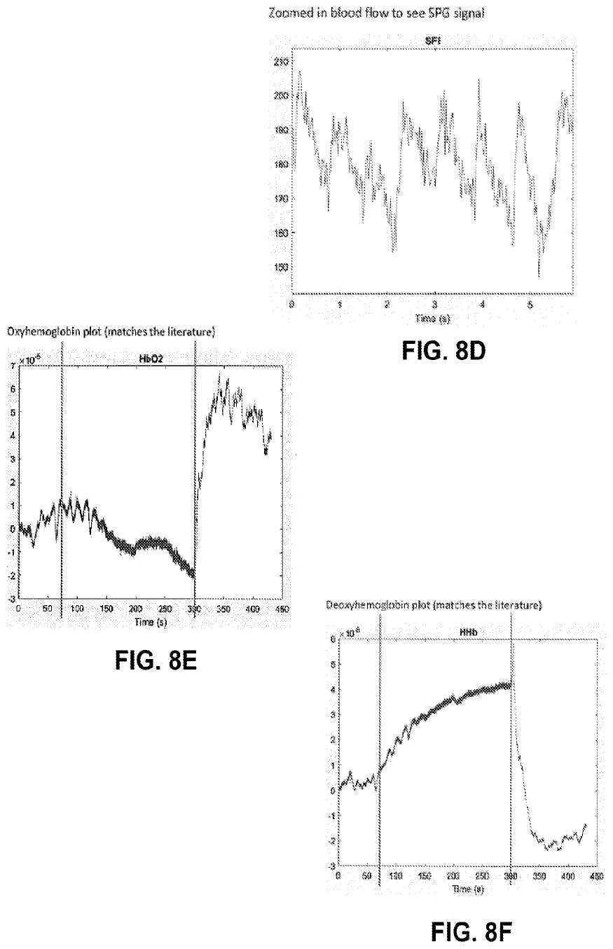

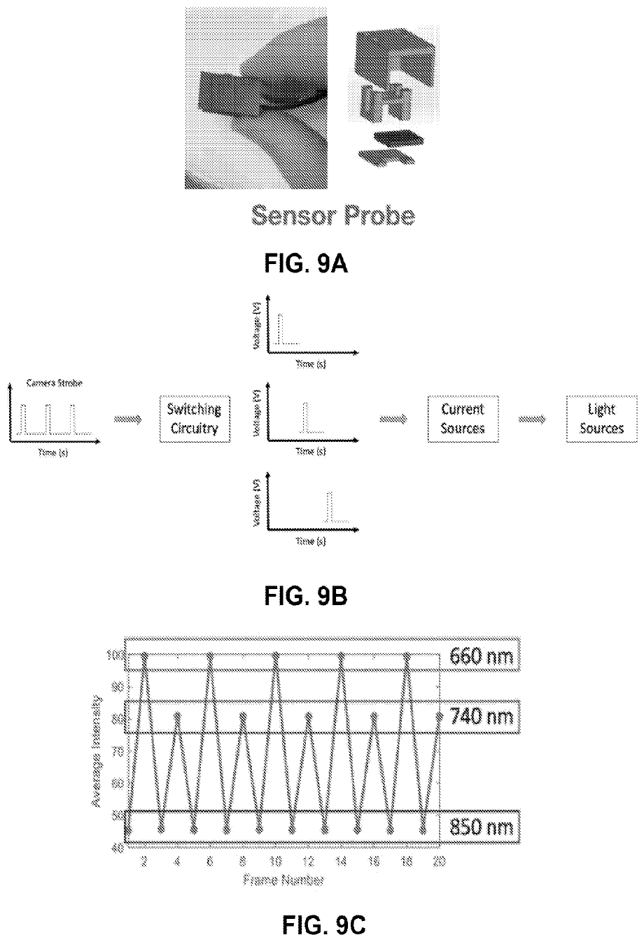

### DETAILED DESCRIPTION OF THE INVENTION

Following is a list of elements corresponding to a particular element referred to herein:

| | |
|---|---|
| 100 | detector |
| 150 | aperture |
| 200 | light sources |

All references, publications, and patents cited herein are incorporated by reference in their entirety as though they are fully set forth. Unless defined otherwise, technical and scientific terms used herein have the same meaning as commonly understood by one of ordinary skill in the art to which this invention belongs. Hornyak, et al., Introduction to Nanoscience and Nanotechnology. CRC Press (2008); Singleton et al., Dictionary of Microbiology and Molecular Biology 3rd ed., J. Wiley & Sons (New York, NY 2001); March, Advanced Organic Chemistry Reactions, Mechanisms, and Structure 7th ed., J. Wiley & Sons (New York, NY 2013); and Sambrook and Russel, Molecular Cloning: A Laboratory Manual 4th ed., Cold Spring Harbor Laboratory Press (Cold Spring Harbor, NY 2012), provide one skilled in the art with a general guide to many of the terms used in the present application. One skilled in the art will recognize many methods and materials similar or equivalent to those described herein, which could be used in the practice of the present invention. Indeed, the present invention is in no way limited to the methods and materials described.

The present invention features a coherent spatial imaging (CSI) system that combines the optical property measurements of srDRS with SPG-based blood flow measurements. This CSI technique allows high-speed measurements of speckle contrast and diffuse reflectance, which provide critical information to assess absolute measurements of pulsatile blood flow, blood volume, stO₂, and tMRO₂. Using miniaturized optoelectronic components allows the present invention to map radially-varying diffuse reflectance of coherent light from tissues, which in turn enables absolute measurement of absorption and scattering contrast. As the supply an

In one embodiment, a device may be used for physiological examination of a subject, comprising a detector (100) and a light source. In another embodiment, the device comprises a detector (100) and a plurality of light sources (200). In another embodiment, the detector (100) comprises a camera with a lens. In another embodiment, the detector (100) comprises a lens that enables a wider field of view but does not require an image to be in focus. In another embodiment, the light source is a coherent light source. In another embodiment, the plurality of light sources (200) comprises a combination of multiple light sources (including, but not limited to LEDs and VCSELs). In another embodiment, the detector (100) includes an aperture (150). In another embodiment, the aperture (150) provides point estimation of optical properties for the device. In another embodiment, the aperture (150) is located in the front of the detector (100). In another embodiment, the aperture (150) is a slit. In another embodiment, the slit has dimensions of about 7 mm long and 0.7 mm wide. In another embodiment, the slit has dimensions of between 2 mm to 12 mm long and 0.2 mm to 2.75 mm wide. In another embodiment, the aperture (150) is a hole. In another embodiment, the device further comprises circuitry. In another embodiment, the device further comprises circuitry to allow rapid switching between wavelengths of the plurality of light sources (200). In some embodiments, the plurality of light sources (200) are at a plurality of different wavelengths and/or similar wavelengths. In another embodiment, the detector (100) comprises two or more holes for placement of light sources (200). In another embodiment, the detector (100) may be modified to provide additional holes for additional placement of light sources (200) for improvement of stability and/or accuracy of the device. In another embodiment, the detector (100) may be modified to provide fewer holes so that the device may be smaller and/or miniaturized. In another embodiment, additional holes are placed for identification of the operating light source at any time point during data collection. In another embodiment, the device has been configured to be used as a sensor that is inexpensive to manufacture, noninvasive, wearable for a user, and/or configured for mobile use. In another embodiment, the device may provide measurements of blood flow, oxygenation, and/or metabolism. In another embodiment, the device further comprises a program for real-time processing of collected images.

Various embodiments herein include use of an aperture that is in the shape of a slit. However, it is not the intent to limit apertures to the slit form only. In one embodiment, for example, the slit aperture allows for greater use of the sensor width along the perpendicular axis for spatially resolved diffuse reflectance spectroscopy. As apparent to one of skill in the art, there are any number of aperture shapes possible that may be effectively used in conjunction with various embodiments herein, and the various embodiments herein that include reference to apertures are in no way limited to only apertures that are in a slit form or configuration.

In one embodiment, a device for physiological examination includes examination of one or more clinical markers. In another embodiment, the device may provide measurements of blood flow, oxygenation, and/or metabolism. In another embodiment, the device comprises a detector (100) operably linked to a plurality of light sources (200). In another embodiment, the detector (100) has an aperture (150). In another embodiment, the detector (100) has an aperture (150) that is in a slit configuration. In one embodiment, the one or more clinical markers comprise tissue metabolic rate of oxygen consumption (tMRO2).

As apparent to one of skill in the art, tMRO2 is a clinical marker for many pathologies such as peripheral arterial disease, cancer, and overall cardiovascular health. Thus, the various embodiments of the device and related methods may also be used for the monitoring, detection, diagnosing, prognosing, and treatment of various pathologies including peripheral arterial disease, cancer, and overall cardiovascular health. Similarly, as apparent to one of skill in the art, there are any number and variety of clinical markers that may be used for examination of physiological examination of a subject, and thus any number of clinical markers may be targeted and/or examined in conjunction with various embodiments of devices and related methods herein and is in no way intended to be only limited to tMRO2 as clinical markers. Similarly, tMRO2 is associated with various diseases and conditions that are in addition to peripheral arterial disease, cancer, and overall cardiovascular health, and various embodiments herein that include tMRO2 as a clinical marker are in no way intended to be limited to only the diseases and/or conditions of peripheral arterial disease, cancer, and overall cardiovascular health.

In one embodiment, a method of diagnosing a disease and/or condition in a subject, comprising providing a device for physiological examination of a subject comprising a detector (100) with an aperture (150) operably linked to a plurality of light sources (200), and diagnosing the disease in the subject by examining one or more clinical markers. In another embodiment, the one or more clinical markers include tMRO2. In another embodiment, the device may provide measurements of blood flow, above-cited oxygenation, and/or metabolism. In another embodiment, the combination of blood flow, optical properties, and oxygenation enables estimation of tissue metabolic rate of oxygen consumption. In another embodiment, the disease and/or condition is peripheral arterial disease, cancer, and/or overall cardiovascular health. In another embodiment, the aperture (150) is a slit. In another embodiment, the aperture (150) provides point estimation of optical properties for the device. In another embodiment, the plurality of light sources (200) comprises three different wavelength sources (including, but not limited to LEDs or vertical-cavity surface-emitting lasers (VCSELs)).

As apparent to one of skill in the art, VCSELs are just one of several possible light sources, and chip-based light sources, that are available and/or may be used. Thus, while various embodiments described herein may also include use of a VCSEL as a possible light source, the various devices and methods described herein are in no way only limited to use of a VCSEL as the light source.

In one embodiment, a method is provided for prognosing a disease and/or condition in a subject, by providing a device for physiological examination of a subject comprising a detector (100) with an aperture (150) operably linked to a plurality of light sources (200), and prognosing a severe and aggressive form of the disease and/or condition by examining one or more clinical markers. In another embodiment, the one or more clinical markers include tMRO2. In another embodiment, the device may provide measurements of blood flow, oxygenation, and/or metabolism. In another embodiment, the combination of blood flow, optical properties, and oxygenation enables estimation of tissue metabolic rate of oxygen consumption. In another embodiment, the disease and/or condition is peripheral arterial disease, cancer, and/or overall cardiovascular health. In another embodiment, the device provides both absolute and relative measurements of blood flow, oxygenation, and tMRO2, instead of only relative measurements. In another embodiment, the aperture (150) is a slit. In another embodiment, the aperture (150) provides point estimation of optical properties for the device. In another embodiment, the plurality of light sources (200) comprises three different wavelength sources, including (but not limited to) LEDs and vertical-cavity surface-emitting lasers (VCSELs).

In one embodiment, a method is provided for treating a disease and/or condition in a subject, by providing a device for physiological examination of a subject comprising a detector (100) with an aperture (150) operably linked to a plurality of light sources (200), and treating the disease in the subject based on the presence of one or more clinical markers. In another embodiment, the one or more clinical markers include tMRO2. In another embodiment, the device may provide measurements of blood flow, oxygenation, and/or metabolism. In another embodiment, the combination of blood flow, optical properties, and oxygenation enables estimation of tissue metabolic rate of oxygen consumption. In another embodiment, the disease and/or condition is peripheral arterial disease, cancer, and/or overall cardiovascular health. In another embodiment, the aperture (150) is a slit. In another embodiment, the aperture (150) provides point estimation of optical properties for the device. In another embodiment, the plurality of light sources (200) comprises three different wavelength sources, including (but not limited to) LEDs and vertical-cavity surface-emitting lasers (VCSELs).

In another embodiment, a device is provided comprising the combination of one or more coherent light sources and a camera detector (100) with a lens in a sensor package. In another embodiment, the sensor is configured to provide images that enable blood flow, oxygenation, and/or metabolism measurements. In another embodiment, the sensor package is placed in close proximity with tissue of a subject, and then images are continuously acquired and/or analyzed for output of physiological parameters. In another embodiment, the presence of tMRO2 is calculated from the measurement of physiological parameters. In another embodiment, the device provides both absolute and relative measurements of blood flow, oxygenation, and tMRO2, instead of only relative measurements.

In another embodiment, the device has a slit placed in front of a camera module to create a pattern that can be processed using a multitude of techniques. In another embodiment, the slit acts as an aperture (150) along one axis to enlarge laser speckles and improve speckle contrast measurements. In another embodiment, the slit allows for greater use of the sensor width along the perpendicular axis for spatially resolved diffuse reflectance spectroscopy. In another embodiment, the combination of blood flow, optical properties, and oxygenation enables estimation of tissue metabolic rate of oxygen consumption. In another embodiment, the device provides rapid data acquisition with the camera to enhance the pulsatile waveform signal quality. In another embodiment, the device provides both absolute and relative measurements of blood flow, oxygenation, and tMRO2 instead of only relative measurements. In another embodiment, the device further comprises a program for real-time processing of collected images.

In one embodiment, a method is provided for modifying the device for physiological examination by providing additional holes in the device for additional placement of one or more light sources for improvement of stability and/or accuracy of the device. In another embodiment, a method is provided for making the device smaller and/or miniaturized by providing fewer holes and/or removing holes in the device.

Embodiments of the present disclosure are further described in the following examples. The examples are merely illustrative and do not in any way limit the scope of the invention as claimed.

In one embodiment, a combination of coherent light sources and a camera detector (100) with a lens into a sensor package. The unique packaging of the sensor forms images that enable blood flow, oxygenation, and metabolism measurements. The sensor package is placed in close proximity with the tissue and then images are continuously acquired and analyzed for output of the physiological parameters. tMRO2 is calculated from the measurement parameters.

In one embodiment, the device provides advantages because it utilizes a slit placed in front of a camera module to create a pattern that can be processed using a multitude of techniques. The slit acts as an aperture (150) along one axis to enlarge the laser speckles and improve the speckle contrast measurements. The aperture (150) in the form of a slit also allows for complete use of the sensor width along the perpendicular axis for spatially resolved diffuse reflectance spectroscopy. In one embodiment, the combination of blood flow, optical properties, and oxygenation enables estimation of tissue metabolic rate of oxygen consumption.

In one embodiment, the device provides advantages over other available but limited in effectiveness alternatives, in that it does not require a high coherence light source, which makes the device significantly less expensive. Furthermore, in one embodiment, the slit eliminates the need for an expensive fiber optic plate and enables simultaneous acquisition of multiple parameters. In another embodiment, the components are also compatible with miniaturization, which reduces bulkiness and increases convenience and usability when compared to some of the other available but limited alternatives.

Further, in one embodiment, rapid data acquisition with the camera can enhance the pulsatile waveform signal quality, when compared to other available but limited alternatives. In one embodiment, the device provides both absolute and relative measurements of blood flow, oxygenation, and tMRO2, instead of only relative measurements.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features, and steps discussed above, as well as other known equivalents for each such element, feature, or step, can be mixed and matched by one of ordinary skill in this art to perform methods in accordance with principles described herein. Among the various elements, features, and steps, some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the selection of constituent modules for the inventive compositions, and the diseases and other clinical conditions that may be diagnosed, prognosed, chip-based therewith. Various embodiments of the invention can specifically include or exclude any of these variations or elements.

In some embodiments, the terms “a,” “an,” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

The reference numbers recited in the below claims are solely for ease of examination of this patent application, and are exemplary, and are not intended in any way to limit the scope of the claims to the particular features having the corresponding reference numbers in the drawings.

1. A device for physiological examination of a subject, comprising:
a detector (100) coupled with a plurality of light sources (200),
wherein the detector (100) has an aperture (150).

2. The device of claim 1, wherein the aperture (150) provides point estimation of optical properties for the device.

3. (canceled)

4. The device of claim 1, wherein the detector (100) comprises a lens that enables a wider field of view but does not require an image to be in focus.

5. The device of claim 1, wherein the plurality of light sources (200) comprise coherent light sources, chip-based light sources, vertical-cavity surface-emitting lasers (VCSELs), light-emitting diodes (LEDs), or a combination thereof.

6-8. (canceled)

9. The device of claim 1, wherein the plurality of light sources (200) are at different wavelengths.

10-11. (canceled)

12. The device of claim 1, wherein the aperture (150) is located in the front of the detector (100).

13. The device of claim 1, wherein the aperture (150) is a slit.

14. The device of claim 1, wherein the aperture (150) is a slit with dimensions of about 2 to 12 mm long and 0.2 to 2.75 mm wide.

15-20. (canceled)

21. The device of claim 1, wherein physiological examination includes examination of one or more clinical markers, measurements of blood flow, oxygenation, and/or metabolism, or a combination thereof.

22. The device of claim 1, wherein physiological examination provides measurements of blood flow, oxygenation, deoxyhemoglobin, oxyhemoglobin, and/or metabolism.

23. The device of claim 1, wherein the device is a wearable.

24. The device of claim 1, wherein the device is miniaturized.

25. The device of claim 1, wherein the device is configured for mobile use.

26. The device of claim 1, wherein physiological examination includes examination of tissue metabolic rate of oxygen consumption (tMRO2) as a clinical marker.

27. A method of diagnosing a disease and/or condition in a subject, comprising:
providing a device for physiological examination of a subject comprising a detector (100) with an aperture (150) operably linked to a plurality of light sources (200); and
diagnosing the disease in the subject by examination of one or more clinical markers in the subject.

28. The method of claim 27, wherein the one or more clinical markers include tMRO2.

29. The method of claim 27, wherein the device provides measurements of blood flow, oxygenation, and/or metabolism and estimation of tissue metabolic rate of oxygen consumption.

30. (canceled)

31. The method of claim 22, wherein estimation of tissue metabolic rate of oxygen consumption is provided by examining the measurements of the combination of blood flow, optical properties, and oxygenation.

32-33. (canceled)

34. The method of claim 27, wherein the aperture (150) provides point estimation of optical properties for the device.

35. (canceled)

36. The method of claim 27, wherein the plurality of light sources (200) are a combination of three different wavelength LEDs and VCSELs.
